(19) United States
(12) Patent Application Publication
(10) Pub. No.: US 2012/0244205 A1
(43) Pub. Date: Sep. 27, 2012

(54) Nanotechnological Delivery of Microbicides and Other Substances

(75) Inventors: Kathleen K. Treseder, Irvine, CA (US); Matthew D. Whiteside, Springdale, AR (US)

(73) Assignee: THE REGENTS OF THE UNIVERSITY OF CALIFORNIA, Oakland, CA (US)

(21) Appl. No.: 13/392,346

(22) PCT Filed: Aug. 25, 2010
(86) PCT No.: PCT/US10/46663

§ 371 Date: Jun. 8, 2012

Related U.S. Application Data
(60) Provisional application No. 61/236,742, filed on Aug. 25, 2009.

(51) Int. Cl. A01N 25/28; A01P 3/00; A01N 43/54; A01N 43/647; B82Y 5/00

(52) U.S. Cl. 424/419; 424/417; 514/269; 514/383; 977/774; 977/773; 977/788; 977/902; 977/810

(57) ABSTRACT

Nanoparticle (e.g., quantum dot) compositions and methods for delivery of substances (e.g., microbicies, fungicides, pesticides, therapeutic agents, biologics, diagnostic agents, dyes, marker substances or tags, etc.) to desired locations within plants or animals. In some embodiments, the quantum dot or naoparticle may be substantially free of cadmiun. In some embodiments, the methods may be employed to prevent to treat fungus or fungus-like infections in pains, such as agricultural crops.

### RELATED APPLICATIONS

This application is a 35 U.S.C. §371 national stage of PCT International Patent Application No. PCT/US2010/046663 filed Aug. 25, 2010 which claims priority to U.S. Provisional Patent Application No. 61/236,742 filed Aug. 25, 2009, the entire disclosure of which is expressly incorporated herein by reference.

### STATEMENT REGARDING GOVERNMENT SUPPORT

This invention was made with United States Government support under Grant No. DEB-0445458 awarded by the National Science Foundation. The United States Government has certain rights in the invention.

### FIELD OF THE INVENTION

The present invention relates generally to agreculture, delivery of microbicidal agents and nanotechnology. More particularly, the present invention relates to compositions and methods wherein nanoparticles, such as quantum dots, are used for the delivery of fungicides and other microbicidal agents.

### BACKGROUND OF THE INVENTION

Pursuant to 37 CFR 1.71(e), this patent document contains material which is subject to copyright protection. The copyright owner has no objection to facsimile reproduction of the entire patent document or the patent disclosure, as it appears in the Patent and Trademark Office patent file or records, but otherwise reserves all copyright rights whatsoever.

Semiconductors are a class of materials. In general, semiconductors are insulators at absolute zero temperature but conduct electricity in a limited way at room temperature. Semiconductor materials can be doped with other substances to alter their electronic properties in a controllable way. Traditional semiconductor materials are crystalline inorganic solids. These materials can be classified according to the periodic table groups from which their constituent atoms come.

The term “nanoparticle” typically refers to particles that have at least one dimension that is 100 nanometers or less. The properties of many conventional materials have been shown to change when formed as nanoparticles. Such changes in material properties are believed to be due to the fact that nanoparticles have a greater surface area per weight than larger particles, thereby rendering them more reactive to certain other substances. Various types of nanoparticles have been devices, including quantum dots, iron nanoparticles, magnetic or paramagnetic nanoparticles, ceramic nanoparticles, nanocrystalline silicon, colloidal gold, carbon nanotubes, silver nanoparticles, silicon nanoparticles, and dendrites.

Quantum dots (sometimes referred to herein as “QDs”) are nanoparticles formed of semiconductor material. More specifically, quantum dots may be nanocrystals composed of semiconductor materials from periodic groups of II-VI, III-V, or IV-VI. Quantum dots are small, typically ranging in diameter from 2-10 nanometers (10-50 atoms).

Fungus infestation or fungi can be cause serious damage to many agricultural crops and/or fungal infections in animals. Examples of phytopathogenic fungi known to infest vegetable crops or other foliage include, but are not limited to those of the classes Ascomycetes and Basidiomycetes. Examples of Ascomycetes include Fusarium, Thielaviopsis, Verticillium, Penicillium and Magnaporthe grisea. Examples of Basidiomycetes include Rhizoctonia, Phakospora pachyrhizi and Puccinia. In addition, oomycetes are not true fungi but are fungal-like organisms. Oomycetes include some highly destructive plant pathogens including those of the genus Phytophthora (e.g., Pythium and Phytophthora). Oomycetes are known to exhibit infection strategies similar to true fungi and, as a result, many plant pathologists group them with fungal pathogens. Thus, as used in this patent application, general or non-specific references to phytopathogenic fungi shall include, but shall not necessarily limited to, all fungi and fungus-like organisms known to infect plants including Ascomycetes, Basidiomycetes and Oomycetes.

Fungicides are chemical compounds or biological organisms used to kill or inhibit fungi or fungal spores. In agriculture, fungicides are used to treat fungal infestations of crops and fungal infections in animals. Fungicides fall into two general categories, contact or systemic. Contact fungicides are applied directly to fungi (e.g., on the surface of a plant) while systemic fungicides must be absorbed by the affected organism.

Fungicides used to treat or deter fungal infestation of commercial vegetable crops include, but are not necessarily limited to: dimethomorph; dimethomorph+mancozeb; fosetyl-aluminum; metalaxl; azoxystrobin; mefenoaxm; potassium bicamonate; copper sulfate; PCMB; DCNA; chlorothalonil; pyraclostrobin; copper oxychloride sulfate; captan; copper hydroxide; copper ammonium carbonate; copper sulfate; mancozeb+copper sulfate; copper sulfate; cymoxanil; mancozeb; chlorothalonil; boscalid; trifloxystrobin; tebuconazole; dimethomorph; mono-, dibasic sodium salts of phosphorus acid; zoxamide/mancozeb; pyraclostrobin; iprodione; copper hydroxide; bacillus subtilis; sulfur; maneb; copper hydroxide/mancozeb; mancozeb; fludioxonil; thiabendazole; sulfur; myclobutanil; copper hydroxide; mancozeb; mono-, dibasic sodium, potassium, and ammonium phosphites; fluopicolide; propamocarb; boscalid+pyraclostrobin; triflumizole; mono-, dibasic sodim, potassium and ammonium phosphites; propiconazole; azoxystrobin; azoxystrobin/chlorothalonil; quinoxyfen; azoxystrobin/propiconzanole; fenamidone; mandipropamid; mandipropamid/difenoconazole; mefenoxam/macozeb; mefenoxam/copper hydroxide; mefenoxam/PCNB; mefenoxam; mefenoxam; vinclozolin; iprodine; pyrimethanil; Bacillus subtillis; Gliocladium virens; sulfur; cypodinil/fludioxonil; metalaxyl+PCNB+Bacillus subtillis; famoxadone+cymoxanil; famoxadone+cymoxanil; dichloropropene/chloropicrin; copper salts of fatty & rosin acid; PCNB; Sulfur; Thiram; Propiconazole; thiophanate-methyl; nemm oil; Mefenoxam; metam-sodium and ziram.

### SUMMARY OF THE INVENTION

The present invention provides novel nanoparticle compositions and methods for delivery of substances (e.g., microbicies, fungicides, pesticides, therapeutic agents, biologics, diagnostic agents, dyes, marker substances or tags, etc.) to desired locations.

In accordance with one aspect of the present invention, there are provided quantum dots or nanoparticles that enhance uptake of certain substances (e.g., fungicides) into certain target cells or organisms (e.g., fungal cells).

Further in accordance with the present invention, there are provided quantum dots or nanoparticles that will 1) facilitate uptake of a substance, such as a microbicidal substance (e.g., a fungicide) by an organism, 2) facilitate subsequent distribution, penetration or transport of the substance into target somatic or non-somatic cells (e.g., fungal cells which infect the organism) and 3) facilitate unpacking of the substance after entry into the target cells.

Still further in accordance with the present invention, there are provided quantum dots or nanoparticles that will selectively deliver a substance (e.g., fungicide) to active sites within target cells (e.g., fungal cells) while deterring uptake of the substance by other cells, such as normal somatic cells of the host organism or other non-target cells or organisms. In this manner, the present invention may be used to increase the efficiency of fungicides or other substances (e.g., pesticides, drugs, biologics, therapeutic agents, etc.) while reducing their undesirable toxicities or untoward effects.

Still further in accordance with the present invention, there are provided substance delivering quantum dots and nanoparticles that are relatively inexpensive to manufacture in bulk quantities and which remain stable in solution for at least a year (the longest-running test to date).

Still further in accordance with the present invention, any suitable type of quantum dot or nanoparticle may be used in combination with any suitable microbicidal substance. In embodiments where the microbicidal substance comprises a fungicide, the fungicide may or may not be selected from the group consisting of: dimethomorph; dimethomorph+mancozeb; fosetyl-aluminum; metalaxl; azoxystrobin; mefenoaxm; potassium bicamonate; copper sulfate; PCMB; DCNA; chlorothalonil; pyraclostrobin; copper oxychloride sulfate; captan; copper hydroxide; copper ammonium carbonate; copper sulfate; mancozeb+copper sulfate; copper sulfate; cymoxanil; mancozeb; chlorothalonil; boscalid; trifloxystrobin; tebuconazole; dimethomorph; mono-, dibasic sodium salts of phosphorus acid; zoxamide/mancozeb; pyraclostrobin; iprodione; copper hydroxide; bacillus subtilis; sulfur; maneb; copper hydroxide/mancozeb; mancozeb; fludioxonil; thiabendazole; sulfur; myclobutanil; copper hydroxide; mancozeb; mono-, dibasic sodium, potassium, and ammonium phosphites; fluopicolide; propamocarb; boscalid+pyraclostrobin; triflumizole; mono-, dibasic sodim, potassium and ammonium phosphites; propiconazole; azoxystrobin; azoxystrobin/chlorothalonil; quinoxyfen; azoxystrobin/propiconzanole; fenamidone; mandipropamid; mandipropamid/difenoconazole; mefenoxam/macozeb; mefenoxam/copper hydroxide; mefenoxam/PCNB; mefenoxam; mefenoxam; vinclozolin; iprodine; pyrimethanil; Bacillus subtillis; Gliocladium virens; sulfur; cypodinil/fludioxonil; metalaxyl+PCNB+Bacillus subtillis; famoxadone+cymoxanil; famoxadone+cymoxanil; dichloropropene/chloropicrin; copper salts of fatty & rosin acid; PCNB; Sulfur; Thiram; Propiconazole; thiophanate-methyl; nemm oil; Mefenoxam; metam-sodium and ziram.

Still further aspects and details of the present invention will be understood upon reading of the detailed description, examples and claims set forth herebelow.

### BRIEF DESCRIPTION OF THE DRAWINGS

FIG. 1A is a schematic diagram of a quantum dot having a polymer coating that contains receptor molecules comprising amino groups which allow the quantum dot to form strong covalent bonds with carboxyl groups of organic compounds.

FIG. 1B schematic diagram of a quantum dot having a polymer coating that contains receptor molecules comprising carboxyl groups which allow the quantum dot to form strong covalent bonds with amino groups of organic compounds.

FIG. 1C is a schematic diagram of a quantum dot that is amino-bound to glycine.

FIG. 1 is a schematic diagram of a quantum dot that is carboxyl-bound glycine.

FIG. 1E is a schematic diagram of a quantum dot that is amino-bound chitosan.

FIG. 1F is a schematic diagram of a quantum dot that is amino-bound arginine.

FIGS. 2A through 2F show superimposed (white light and fluorescence) confocal laser scans of Peniciffium solitum uptake of QD-ON conjugates taken at various time points during incubation with different conjugates. In FIG. 3A (taken at 2 hrs) fungal hyphae show evidence of uptake. In FIG. 3B (taken at 6 hrs) less labeled glycine is seen in solution and more is seen within the hyphae. In FIG. 3C (taken at 24 hrs) very little labeled glycine is seen outside of the hyphae. In FIG. 3D (taken at 24 hrs) QD-arginine fluorescence appears in the cytoplasm but not in vesicles (arrows). In FIG. 3E (taken at 5 hours) uptake of QD labeled chitosan is evident. FIG. 3F shows a QD control (quantum dots unbound to glycine) after 24 hrs of incubation with no signs of uptake.

FIGS. 3A through 3G show confocal light and emission scans of QD-ON conjugates after incubation with the roots and mycorrhizal colonists of annual bluegrass (Poa annua). FIG. 3A shows fluorescence 4 hrs after incubation with QD-glycine from AMF hyphae. FIG. 3B shows fluorescence 4 hrs after incubation with QD-glycine into the plant root. FIG. 3C shows fluorescence 4 hrs after incubation with QD-glycine and up to the plant shoot. FIG. 3D shows internal fluorescence of AMF vacuoles 24 hrs of incubation with QD-glycine (arrows). FIG. 3E shows fluorescence of vascular tissue of the plant root (arrows) after 24 hrs of incubation with QD-glycine. FIG. 3F shows fluorescence of chloroplasts in the shoot cells (arrows) after 24 hrs of incubation with QD-glycine. FIG. 3G shows fluorescence in AMF after 5 hrs of incubation with QD-chitosan.

FIGS. 4A through 4G show fluorescence of QD-glycine in a plant and fungi using field imaging techniques. Areas of fluorescence indicate presence of QDs. Specifically, FIGS. 4A through 4D are unmagnified digital images of a Poa annua individual incubated with orange excitation QD-glycine. FIG. 5A is a white light view. FIG. 4B is a UV view. FIG. 4C is a view whowing root detail. FIG. 4D is a view showing blade detail. FIG. 4E through 4G are minirhizotron images of fungal hyphae uptake of QD-glycine. FIG. 4E is a white light image pre-QD injection. FIG. 4F is a UV image pre-QD injection. FIG. 4G is taken at 2 hrs after injection of QD-glycine and shows hyphal uptake (arrow) of labeled glycine.

FIGS. 5A through 5C represent raster-scanned false color images of QD-chitosan within intracellular arbuscular mycorrhizal (AM) hyphae and a plant root. Specifically, FIG. 5A represents a confocal scan of an Alaskan root colonized with AM hyphae. FIG. 5B represents a false color image, with warmer colors representing higher fluorescent intensities. FIG. 5C represents an enlarged raster scan (33.8×zoom) of QD-chitosan (616-624 nm emission) within the intracellular AM hyphae of FIG. 5B. An average of 6,241 QD particles per μm³ AM tissue were quantified across the sample image.

FIG. 6 is a graph showing growth inhibition of Rhizoctonia fungi by QDs conjugated to Azoxystrobin plus glycine (“QD+AI”) versus Azoxystrobin alone (“AI alone”). QD conjugation increased effectiveness of the fungicide 10-fold. AI=active ingredient.

FIG. 7 is a graph showing growth inhibition of Rhizoctonia fungi by QDs conjugated to Difenoconozole plus glycine (“QD+AI”) versus Difenoconozole alone (“AI alone”). QD conjugation increased effectiveness of the fungicide 10-fold. AI=active ingredient.

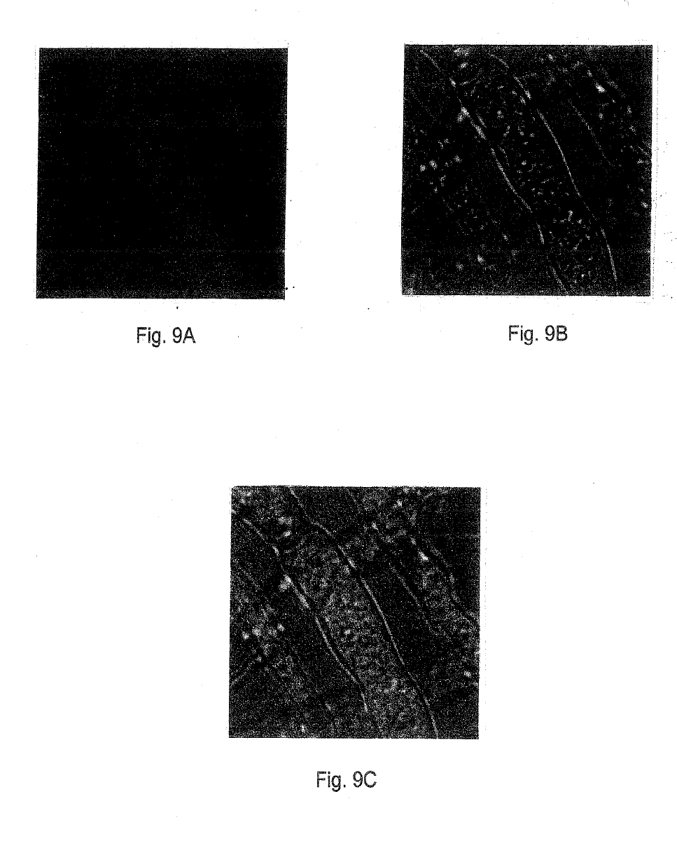

FIG. 8 is a graph showing growth inhibition of Rhizoctonia fungi exposed to QDs conjugated with glycine but no fungicide (“QD controls”), at various time lengths of incubation. Note that negative values of growth inhibition correspond to increases in growth. AI=active ingredient.

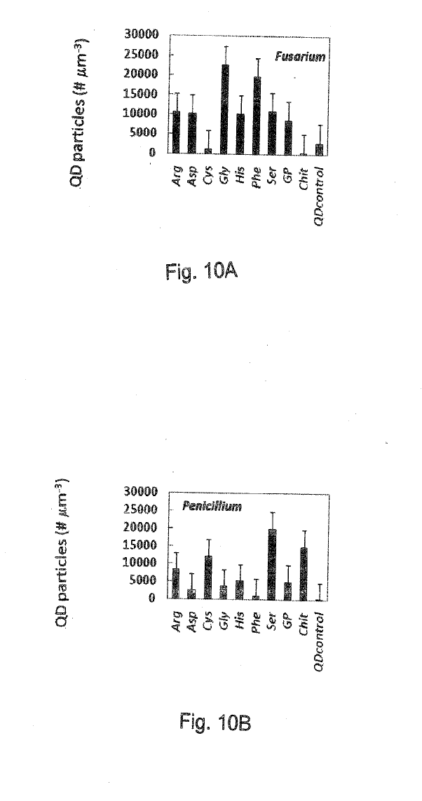

FIGS. 9A through 9C show Fusarium fungi displaying QDs conjugated with glycine and Difenoconazole. Specifically, FIG. 9A represents a fluorescent-light image, FIG. 9B represents a white light image and FIG. 9C represents overlayed fluorescent+white light images. Areas of fluorescence denote presence of a QD conjugated with glycine and Difenoconazole, as confirmed by spectral analysis.

FIGS. 10A through 10D are graphs showing in vitro uptake rates (over a 24-hr incubation period) into cytoplasm of QDs conjugated with one of seven amino acids (I-r, arginine, asparagine, cystein, glycine, histidine, phenylalanine, and serine), chitosan (“Chit”), α-D-glucose-1-phosphate (“GP”) or no targeting probe(QDcontrol). Four evolutionarily-distinct fungi (Fusarium, Penicillium, Rhizoctonia, and Phytophthora) were tested.

FIGS. 11A through 11D are graphs showing in vitro uptake rates (over a 24-hr incubation period) into cytoplasm of QDs conjugated with one of seven amino acids (I-r, arginine, asparagine, cystein, glycine, histidine, phenylalanine, and serine), chitosan (“Chit”), α-D-glucose-1-phosphate (“GP”) or no targeting probe (QDcontrol). Four evolutionarily-distinct fungi (Fusarium, Penicillium, Rhizoctonia, and Phytophthora) were tested.

FIGS. 12A through 12F are graphs showing Mycorrhizal responses under N fertilization. Mycorrhizal fungi were analyzed from the top 10 cm of soil. Uptake was documented in internal AM hyphae and in ECM root tips. For AM fungi, N additions significantly decreased specific uptake of QD-glycine (P=0.006) (FIG. 12A). Nitrogen fertilization had no significant effect on specific uptake (per unit biovolume) of QD-glycine by ECM fungi (FIG. 12B). ECM standing biomass significantly decreased with N fertilization (P=0.004; FIG. 12D). In contrast, AM standing biomass did not (C). At the plot-level, uptake of QD-glycine by AM fungi (per unit land area) significantly declined under N fertilization (P=0.001; FIG. 12E); QD-glycine uptake by ECM fungi did not (FIG. 12F).

FIGS. 13A through 13D are graphs showing Mycorrhizal uptake of QD-chitosan under N fertilization. Nitrogen fertilization had no significant effect on specific uptake of QD-chitosan by AM (FIG. 13A) or ECM fungi (FIG. 13B). However, at the plot-level, uptake of QD-chitosan by ECM fungi (per unit land area) significantly declined under N fertilization (P=0.025; FIG. 13D) In comparison, N fertilization had no significant effect on plot-level uptake of QD-chitosan by AM fungi (FIG. 13C).

### DETAILED DESCRIPTION AND EXAMPLES

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

### Nano-Packaging of Fungicides

Some quantum dots comprise a cadmium selenide core wrapped in a zinc sulfide shell. Such quantum dots may range in diameter from 2 nm to 20 nm. (For comparison, 2 nm is equivalent to the size of two nucleic acids.) The quantum dots can be covered with protective polymer coatings and embedded with receptor molecules that allow the quantum dot to be conjugated with other compounds, including fungicides. In addition, quantum dots of the present invention can optionally be bound to various targeting probes that will induce uptake by the desired target cell or organism (e.g., a fungal pathogen of interest), inhibit uptake by other cells or organisms, and/or direct the quantum dot to an intended site of activity of the substance (e.g., fungicide) within the target cell or organism (e.g., the nucleus, cytoplasm, vesicle, etc.). In this way, quantum dots of the present invention can act as “nano-packages” of desired substances (e.g., fungicides) and target probes that can be fine-tuned to deliver the substances (e.g., fungicide) precisely where needed.

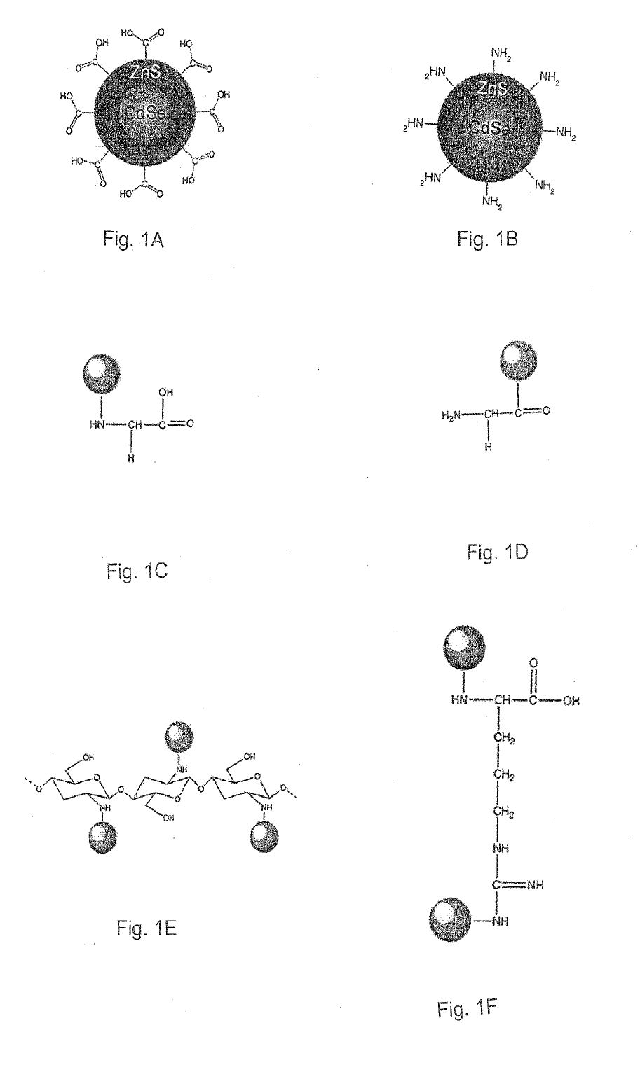

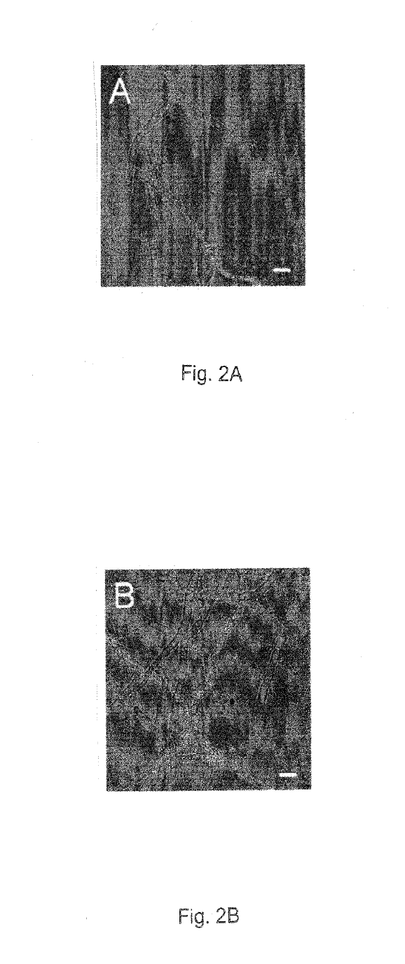

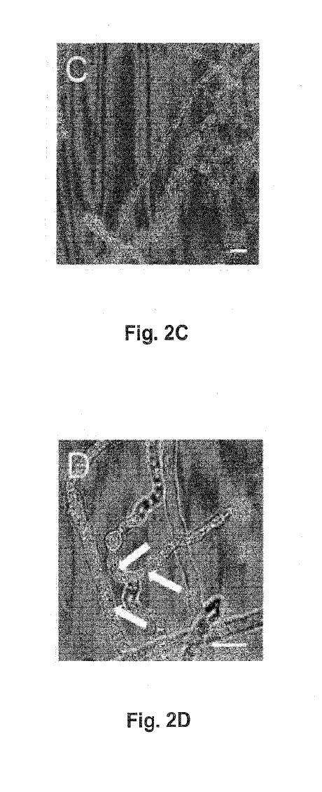

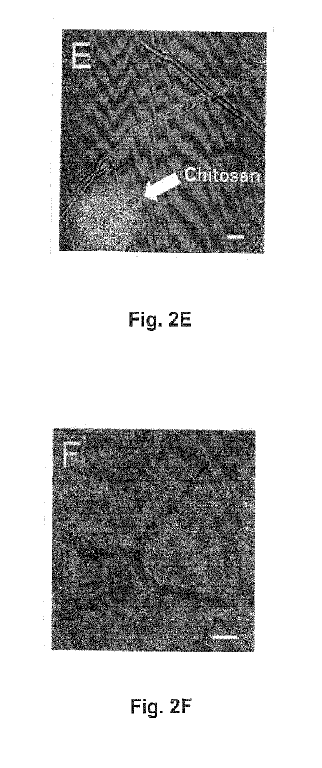

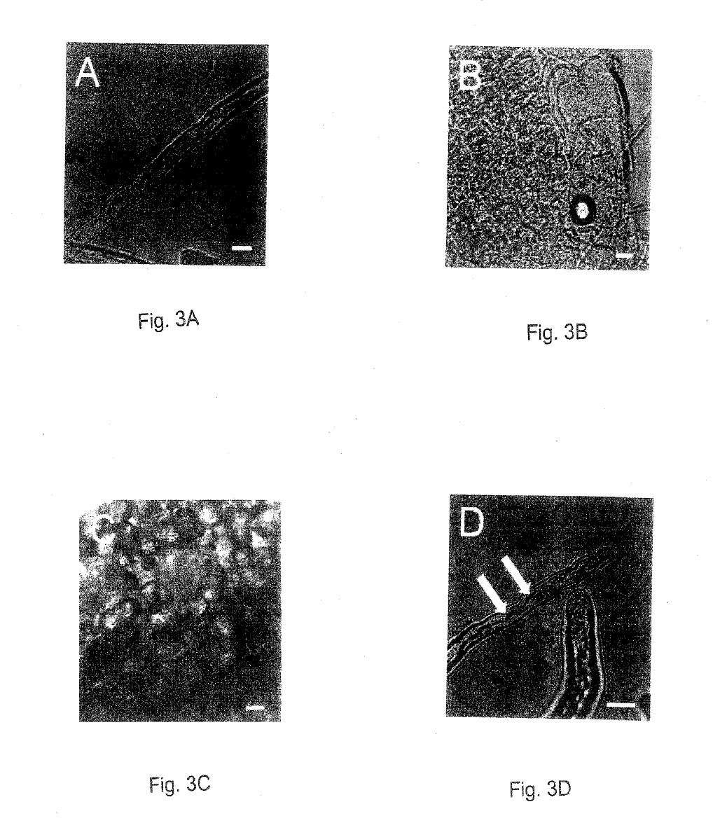

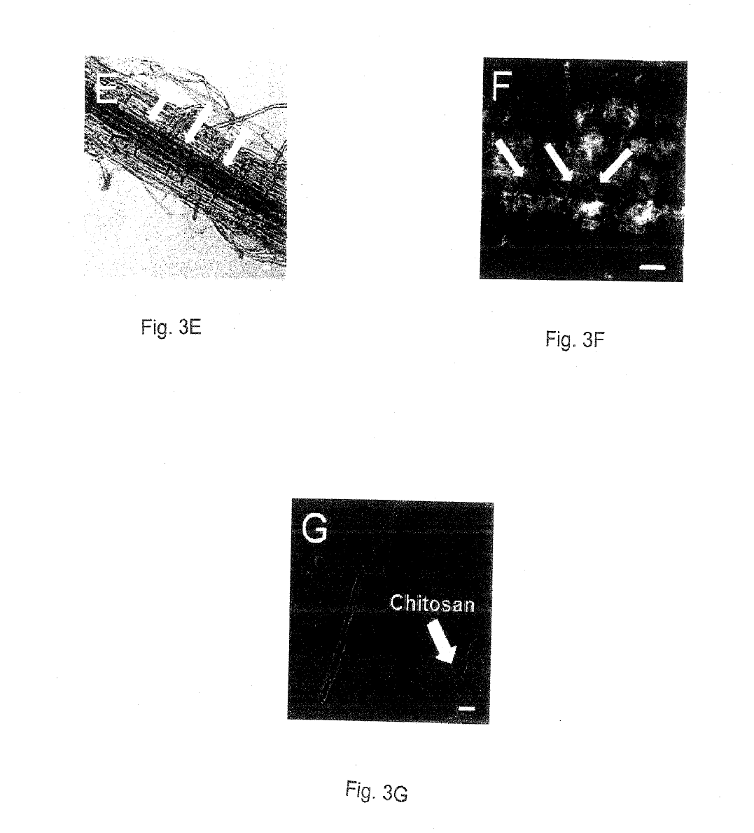

FIGS. 1A and 1B are schematic diagrams of a quantum dots. The polymer coating contains receptor molecules that allow the quantum dot to form bonds with targeting probes and fungicides. For example, receptor molecules can be carboxyl groups, as seen in FIG. 1A, or amino groups as shown in FIG. 1B. The quantum dots of the present invention may be designed to have a specific combination of receptor molecules and targeting probes to optimize effectiveness.

Quantum dots of the present invention can also be designed to release the substance (e.g., fungicide) only after reaching the target location by using combinations of receptor molecules (FIGS. 1A and 1B) that form bonds, attractions or other connections with the substance (e.g., the fungicide) that are strong enough to carry the substance (e.g., fungicide) to the target location, but weak enough that the substance (e.g., fungicide) will be released or “unpacked” at that target location. The combination of receptor molecules and their bound targeting probes can be precisely designed for each fungicide and pathogen or other target.

In at least some embodiments of the invention, the quantum dots can be tracked within the organism. For example, when exposed to ultraviolet light, quantum dots of the present invention will glow in precise colors of almost any wavelength of visible light in sufficient brightness or magnitude to allow the location of the quantum dots to be visualized even when they are inside an a organism such as a fungus or plant. This ability of quantum dots to glow also may be used in research, development or testing how efficiently certain quantum dots deliver their substance(s) (e.g., fungicides) to the appropriate location within the target cell or organism.

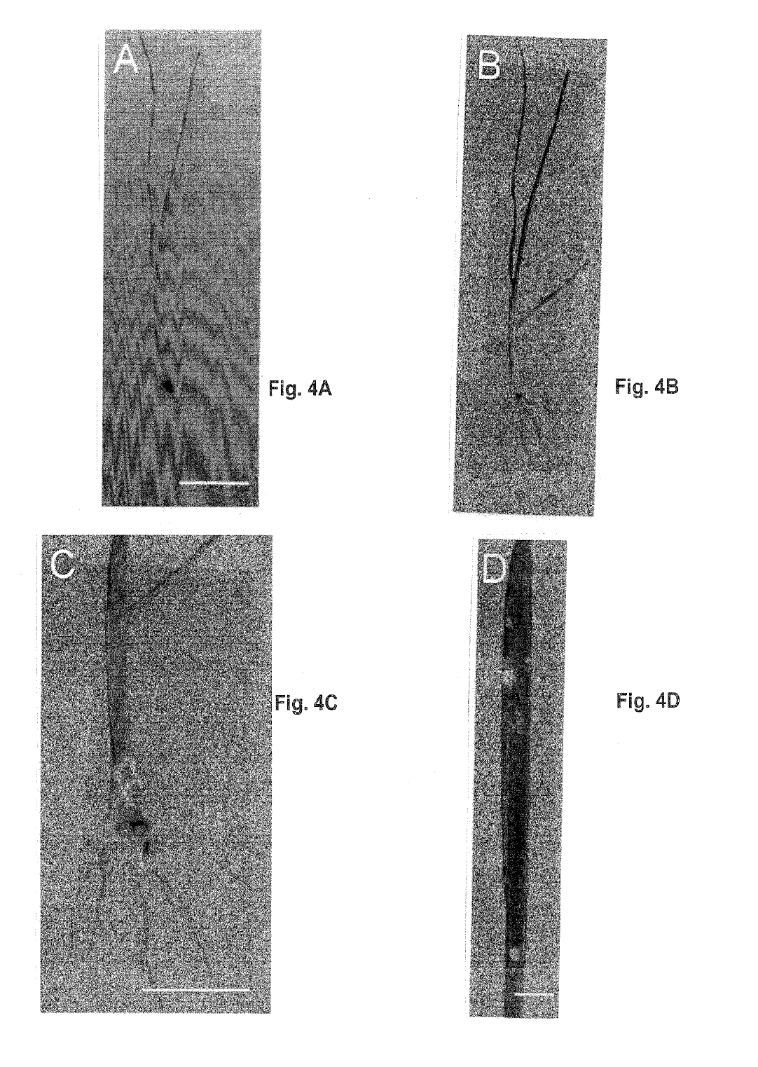

Applicant has performed tests which demonstrate that fungi and plants actively take up and translocate the quantum dot-conjugated amino acids from soil and media. Results of these tests are illustrated in the laser scans of FIGS. 2A through 2F.

Quantum dots of the present invention are readily taken up by fungi when the quantum dots are conjugated with amino acids like glycine and arginine. Moreover, quantum dots of the present invention are directed to different locations within the fungus depending on the type of amino acid to which they are bound. Specifically, glycine-bound quantum dots are translocated to vesicles within fungal cells (FIG. 2c), whereas arginine-bound quantum dots remain in the cytoplasm (FIG. 2d). In other words, these amino acids are serving as targeting probes. In addition, quantum dots conjugated with the amino sugar chitosan were taken up reasonably well by Penicillium fungi (FIG. 2e), but less readily by fungi of the Glomeromycota (i.e., arbuscular mycorrhizal fungi), which form beneficial relationships with plants. In this way, quantum dots are targeting particular species of fungi. To our knowledge, we are one of the first research groups to apply quantum dots to studies of fungi, soils, or plants.

### Quantum Dots Track Organic Nitrogen Through Fungi and Plants

Soil microorganisms mediate many nutrient transformations that are central in terrestrial cycling of carbon and nitrogen. However, uptake of organic nutrients by microorganisms is difficult to study in natural systems. We assessed quantum dots (fluorescent nanoscale semiconductors) as a new tool to observe uptake and translocation of organic nitrogen by fungi and plants. We conjugated quantum dots to the amino groups of glycine, arginine, and chitosan, and incubated them with Penicillium fungi (a saprotroph), and annual bluegrass (Poa annua) inoculated with arbuscular mycorrhizal fungi. As experimental controls, we incubated fungi and bluegrass samples with substrate-free quantum dots as well as unbound quantum dot substrate mixtures. Penicillium fungi, annual bluegrass, and arbuscular mycorrhizal fungi all showed uptake and translocation of quantum dot-labeled organic nitrogen, but no uptake of quantum dot controls. Additionally, we observed quantum dot-labeled organic nitrogen within soil hyphae, plant roots, and plant shoots using field imaging techniques. This experiment is one of the first to demonstrate direct uptake of organic nitrogen by arbuscular mycorrhizal fungi.

Microbial communities mediate many of the soil processes that provide nutrients to plants and that release trace gases to the atmosphere. Thus, understanding how these microbial communities function is essential to understanding ecosystems as a whole. Ecosystem ecologists have traditionally approached soils as a black box in which nutrient transformations are conducted by an uncharacterized community of microorganisms (Tiedje et al. 1999). In many cases, this framework has been appropriate—it has allowed investigators to construct nutrient budgets of ecosystems and to document patterns in soil characteristics. However, we must possess more mechanistic information about the functioning of microorganisms in ecosystems to adequately predict nutrient dynamics under a range of environmental conditions (Treseder and Allen 2000). Historically, detailed investigations have been hampered by limitations inherent in resolving the spatial dynamics of microbial processes taking place in a complex, fragile, and opaque environment.

One recent subject of debate is the extent to which plants are able to directly consume organic nitrogen (ON). Traditionally, ON was thought to undergo a transformation into inorganic N before being taken up by plants (Liebig 1843). This theory assumes N mineralization is the limiting process for plant growth in most ecosystems (Vitousek 1997). However, isotope labeling has provided evidence that plants can take up ON, at least as simple forms such as amino acids (Turnbull et al. 1995, Lipson and Monson 1998, Nasholm et al. 1998, Lipson and Nasholm 2001, Miller and Cramer 2005, Rains and Bledsoe 2007). It is unclear, however, whether ON molecules are directly assimilated by plant roots or if plants acquire ON indirectly via mycorrhizal fungi or other microbial symbionts.

Arbuscular mycorrhizal fungi (AMF) are common worldwide, and form symbioses with approximately 80% of plant families (Newman and Reddell 1987, Smith and Read 1997). They mine the soil for nutrients and translocate a portion to their host plants in exchange for carbon (Smith and Read 1997). The prevailing paradigm is that AMF specialize in the capture of inorganic nutrients such as phosphate, ammonium, and nitrate (Read 1991, Smith and Smith 1997). Uptake of ON by AMF has been challenging to investigate in natural systems because of technical difficulties in tracing the flow of organically-derived nutrients from the soil into hyphae.

One promising technique to investigate the flow of nutrients between plants, microorganisms, and soil in field conditions is the use of quantum dots. Quantum dots (QDs) are nanoscale semiconductors that fluoresce in different colors depending on their size (Chan and Nie 1998, Wong and Stucky 2001, Reiss et al. 2002, Alivisatos et al. 2005). They are typically composed of a cadmium selenide core wrapped in a zinc sulfide shell, and they can be enclosed by protective polymer coatings (FIG. 1) (Dubertret et al. 2002). Polymer coated QDs are commercially available, and range in diameter from 2 nm (NNT, Ontario, Canada) up to 20 nm (Invitrogen, Carlsbad, Calif.). There are many advantages of QDs over traditional labels and tags. Quantum dots have broad absorption spectra with very narrow emission peaks, meaning multiple colors can be assessed on the same sample by using the same light source. Since QDs are resistant to metabolic and chemical degradation and are not susceptible to photobleaching, they are effective tracers for long term studies (Alivisatos et al. 2005, Michalet et al. 2005). Kloepfer et at (2005) found that QD-labeled bacteria (Bacillus subtilis) retained QD fluorescence and electron density, even after storage for one year in rich medium. In addition, QDs can be bound to amino or carboxyl groups within organic compounds (FIG. 1). In this way, the movement of these compounds through soils or organisms can be imaged and tracked (Dubertret et al. 2002, Kloepfer et al. 2005). Previously, most quantum dot work has focused on biomedical applications (Gao et al. 2004, Alivisatos et al. 2005, Garon et al. 2007, Qian et al. 2007). To our knowledge, this study is the first to apply quantum dot technology to ecological questions.

In this example, QDs are used as a tool to observe the translocation of ON in laboratory and soil-based systems. Specifically, our objectives were to use QDs to track ON uptake (labile and recalcitrant) by a model group of non-mycorrhizal fungi, Penicillium solitum, and by AMF-colonized plants. By testing this technique on Penicillium fungi, we assessed uptake of QD-substrates in a known ON-acquiring saprotroph as a proof-of-method. We then extended this method to AMF to examine ON uptake by this group. We used glycine and arginine (common amino acids) to represent labile ON, and chitosan (deacylated form of chitin) to represent recalcitrant ON.

Conjugation of Quantum Dots.

Quantum dot (QD) conjugation was performed by following a modified Kloepfer et al. (2005) method. Commercial red (565 nm emission) carboxyl QDs (Invitrogen, Carlsbad, Calif.) were conjugated with the amino group of reagent-grade glycine or arginine by using the binding activator 1-ethyl-3-(3-dimethylaminopropyl) carbodiimide hydrochloride (EDC). The reaction was performed in a 10 mM borate buffer (pH 7.4). Each mL of reaction solution contained 3.33 mM substrate, 0.1 μM QDs, and 2 mg EDC. The conjugation reaction was performed on a gentle shaker for 3 h in the dark. The quantum dot conjugates underwent dialysis against 2 L of sterile water and were then centrifuged to remove any sedimented material. Quantum dot conjugation of chitosan (MP Biomedical, Solon, Ohio) was performed in the same manner as for glycine and arginine except each mL of reaction solution contained 5.8 mg chitosan, 0.1 μM QDs, and 2.2 mg EDC. The QDs used in this study were less than 15 nm in diameter (Invitrogen, Carlsbad, Calif.).

Uptake of QD-On by Penicillium solitum.

To assess uptake of QD labeled substrates, we tested a common saprotrophic fungus, Penicillium solitum. Penicillium solitum was grown from spores in liquid Modified Melin-Norkrans (MMN) media (Marx 1969). After 24 h, hyphae were transferred with 250 μL MMN into microcentrifuge tubes. The tubes were incubated with either 250 μL (0.1 μM) of QD-glycine in a time series ranging from 2, 6, or 24 h; 250 μL (0.1 μM) of QD-arginine, 24 h; 2.65 mg QD-chitosan in 250 μL sterile water, 5 or 24 h; a QD control of 250 μL (0.1 μM) QDs containing no substrate, 2, 4, 6, or 24 h; or QD controls of 250 μL (0.1 μM) QDs and substrate (glycine, arginine, or chitosan) lacking binding reagent, 2, 4, 6, or 24 h. After incubation, the hyphae were centrifuged and washed with 1% saline to remove unbound QDs. All cultures and incubations were performed in the dark. Additionally, all preceding steps were performed under sterile conditions. Quantum dots were excited using an Argon/2 laser (458 or 488 nm), and fungal uptake was observed with a Zeiss LSM 510 META confocal microscope. The detector was set to 565 nm±10 nm and was calibrated to display no fluorescence of unlabeled material. This approach reduced the incidence of autofluorescence.

Uptake of QD-ON by Arbuscular Mycorrhizal Fungi and Poa annua.

To test uptake of QD-ON substrates by AMF-colonized plants, annual bluegrass (Poa annua) was grown from seed in 1:1 autoclaved sand:vermiculite. Seedlings were cultivated in an 11 L planting pot inoculated with a mixture of AMF (Mycorrhizal Applications, Grants Pass, Oreg.). After 30 d of growth, mycorrhizal plants were transferred to sterile 10 mL capped test tubes containing 3.5 g of autoclaved 1:1 sand and vermiculite and 1.6 mL sterile water. Each tube received one plant. The plants were incubated with 500 μL QD-glycine solution, QD-chitosan, or QD control for either 4 or 24 h. After incubation, the plants were removed and gently washed with 1% siline to help remove sand particles and unbound QDs. Confocal laser scans were performed as described above.

Field-Applicable Imaging.

To test the imaging capabilities of QD-ON substrates under more natural conditions, we used a minirhizotron to detect QD fluorescence in soils. Eight g of Poa annua seed was cultivated in an 11 L planting pot of autoclaved 1:1 sand:vermiculite mix. The pot was inoculated with a multi-species mix of AMF (Mycorrhizal Applications, Grants Pass, Oreg.) and fitted with a horizontal minirhizotron tube. Five glass applicator tubes were installed along the minirhizotron tube so that the ends of the applicators rested ˜2 mm above the minirhizotron tube. The pot was watered twice a day on a misting bench in a 23° C. greenhouse. After 30 d of growth, 25 mL (0.1 μM) of QD-glycine was injected into one applicator tube above an area of observable hyphal growth. Uptake was observed using a BTC-100× minirhizotron camera (Bartz Technology, Santa Barbara, Calif.), which is fitted with an UV light source.

To further test QDs as a non-destructive method that could be applied in the field, we used a handheld UV light source and digital camera to observe unmagnified QD fluorescence in plant shoot tissue. Orange excitation (585 nm emission) carboxyl QDs were bound to the primary amino group of glycine as described above. Poa annua seedlings were grown in 1:1 autoclaved sand and vermiculite as described above. After 20 d, seedlings were transferred to sterile microcentrifuge tubes containing 1 mL QD-glycine (1 μM) solution in liquid MMN media. To maintain the media at a consistent volume during the experiment, liquid MMN was added as needed. After 14 d the seedlings were harvested and thoroughly rinsed with 1% saline solution to remove any QDs unassociated with the plants. The QD conjugates were excited using a 4 v portable handheld UV light, and photos were taken with a Canon 7.1 Mp digital camera without zoom or magnification.

Uptake of QD-ON by Penicillium solitum.

We used Penicillium solitum to test the uptake and storage of unconjugated QDs, QD-glycine, QD-arginine, and QD-chitosan by a common, well-studied saprotrophic fungus. Within 2 h of incubation with QD-glycine, fluorescence became apparent in the vacuole compartments of the hyphae (FIG. 2A). Uptake continued throughout the 24 h duration of the experiment and QD concentrations in hyphae intensified with time (FIG. 2B,C). Uptake was also evident using QD-arginine, but for the 24 h duration of the experiment the label appeared to remain outside the vacuoles and instead stayed within the cytoplasm (FIG. 2D). The P. solitum fungi were able to acquire QD-chitosan (FIG. 2E). Fluorescence was observed in the cytoplasm of the hyphae within 5 h of incubation. Additionally, neither of the QD controls displayed any sign of uptake or surface binding to the hyphal walls (FIG. 2F).

Uptake of QD-ON by Arbuscular Mycorrhizal Fungi and Poa annua.

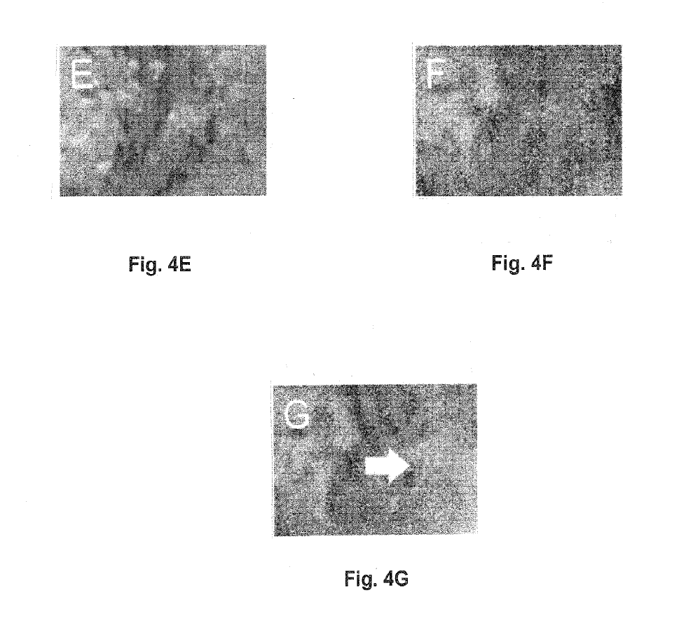

We used individuals of Poa annua inoculated with AMF to track the movement of QD-glycine, QD-chitosan, and QD controls to hyphal, root, and shoot structures. The QD-glycine labels were first observed in AM hyphae (FIG. 3A), plant root cells (FIG. 3B), and plant shoot cells (FIG. 3C) within 4 h of incubation. After 24 h of incubation, QD-glycine had collected in specific structures within each tissue type: tube-shaped vacuoles within AM hyphae (FIG. 3D), vascular tissues within roots (FIG. 3E), and chloroplasts within shoot cells (FIG. 3F). The AM hyphae contained QD-chitosan within 5 h of incubation (FIG. 3G), but no QD fluorescence was observed in the plant roots or shoots during the 24 h QD-chitosan treatment (data not shown). Moreover, no uptake or transportation of any QD control was seen in the fungi, plant roots, or plant shoots (data not shown).

Field-Applicable Imaging.

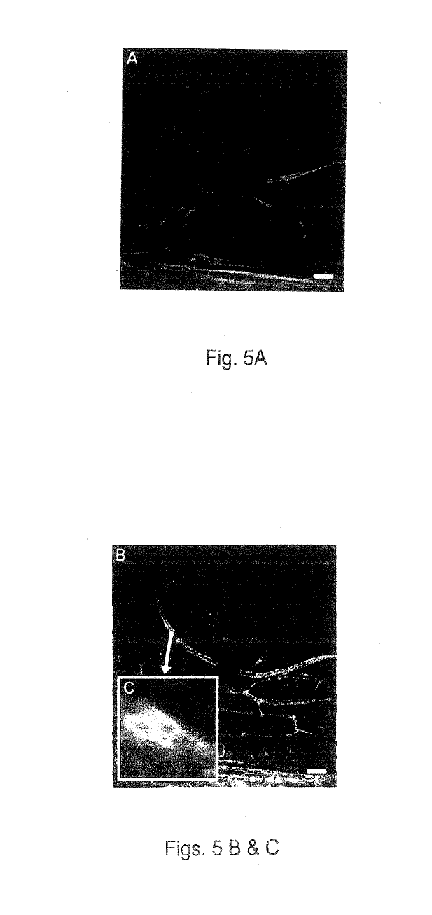

We observed fluorescence of QD-glycine within plant material and fungal hyphae by using imaging techniques that can be applied in field settings. Poa annua fluoresced with QD-glycine in both roots and shoots during a 14 d incubation period (FIG. 4A-D). We were able to excite the QD-glycine with a handheld UV light source and observe movement of the label with the naked human eye. Additionally, we used a minirhizotron camera (4 μm resolution) to track the flux of QD-glycine from soil to fungal hyphae in a greenhouse experiment. The hyphae were only slightly visible before QD-glycine injection, but fluoresced 2 hrs after injection (FIG. 4E-F).

In this example, the QD-labeled ON was taken up and transported to particular structures in fungi and plants, depending on the type of ON compound conjugated to the QD. In natural settings, proteinaceous materials (e.g., amino acids, peptides, and proteins) comprise 40% of soil nitrogen and aminosugars comprise 5-6% (Schulten and Schnitzer 1998). As reviewed by Rentsch et al. (2007) and documented by Paungfoo-Lonhienne et al. (2008), saprotrophic and ectomycorrhizal fungi are known to directly acquire amino acids, peptide chains and proteins and plants contain transporters for amino acids, peptides and proteins. Additionally, transportation of chitin and other polysaccharides is not well studied. Previous studies have shown that Penicillium fungi take up and compartmentalize intact amino acids such as glycine and arginine (Kitamoto et al. 1988, Hillenga et al. 1996) and are capable of breaking down and absorbing more recalcitrant forms of ON such as the polysaccharide chitin (Binod et al. 2007). Penicillium fungi compartmentalize nutrients and amino acids in two main locations: in vacuoles where they can be stored, and in cytoplasm where they can be quickly metabolized or transformed (Griffin 1994). Vacuoles of Penicillium hyphae can contain up to 51% of total cellular glycine and up to 98% of total cellular arginine (Kitamoto et al. 1988, Roos et al. 1997). We observed QD-chitosan in cytoplasm (FIG. 2E) and QD-glycine in vacuoles (FIG. 2A-C), but QD-arginine was observed in cytoplasm and not vacuoles (FIG. 2D). The lack of QD-arginine in vacuoles is most likely the result of QD-arginine size after conjugation. Glycine contains one amino terminal as a potential QD binding site (FIG. 3C), however, arginine contains two (FIG. 1F). Therefore, it is possible for each arginine molecule to bind with more than one QD during conjugation. QD-arginine (theoretically twice as large as QD-glycine) may be small enough to pass through the cell wall, but too large to pass into the vacuole.

Plant roots can acquire amino acids individually or as peptides and proteins via endocytosis and various amino acid, peptide, and protein transporters (Fischer et al. 1998, Rentsch et al. 2007, Paungfoo-Lonhienne et al. 2008). Absorbed proteinaceous material can then be transferred to vascular tissue (phloem and xylem), where it is transported to and from the shoot or transformed into other molecules (Rentsch et al. 2007). In the shoot, amino acids and peptides are exchanged between vascular tissue and mesophyll cells where they are metabolized and assimilated by chloroplasts (Miflin and Lea 1977). Subsequently, chloroplasts contain the largest concentration if amino acids in the shoot (Riens et al. 1991). In our experiment we visually confirmed similar transfers using QD-glycine. We observed the movement of QD-glycine from root cells (FIG. 3B) to vascular tissue (FIG. 3E) and from mesophyll cells (FIG. 3C) to chloroplasts (FIG. 3F) within a 24 h period. If the bound substrates or polymer coatings were removed from QDs by plant or microbial processes, we would expect QD fluorescence in the main storage sites of cadmium (QD core) or zinc (QD shell) within plants. Plants typically store cadmium in the apoplast (free space outside the plasma membrane) and zinc in the vacuoles of epidermal and subepidermal cells (cells lacking chloroplasts) (Vazquez et al. 1992, Vazquez et al. 1994, Sarret et al. 2002). However, in our study we observed QD fluorescence in vacuoles of mesophyll cells and in chloroplasts; areas expected to contain amino acids individually or as peptide chains.

Contrary to the prevailing paradigm, AMF took up labile (QD-glycine) and recalcitrant (QD-chitosan) forms of ON. QD-chitosan was located within the cytoplasm after 5 h of incubation, similar to Penicillium solitum. QD-glycine was located in tube shaped vacuoles, complimenting AMF location of phosphate in previous studies (Ashford 2002, Uetake et al. 2002). As shown in FIG. 1A, the red QDs used were encased in a polymer with carboxyl terminals, so even if the ON attached was mineralized it was still bound to a carboxyl group of the QD polymer. Since QDs were taken up only when bound to ON compounds, it appears that ON was acquired directly and actively by AMF.

A few studies have demonstrated that AMF can obtain N derived from organic sources (Cliquet et al. 1997, Hawkins et al. 2000, Hodge et al. 2001, Rains and Bledsoe 2007), but without direct imaging it is difficult to determine if AMF can take up N in organic form. In a laboratory microcosm, Hodge et al. (2001) placed ¹⁵N-labeled litter in soil compartments that could be accessed by AM hyphae but not by plant roots. In doing so, they demonstrated that AMF can acquire N from organic material. However, this approach cannot be easily replicated under field conditions. Mesh-enclosed cores could be installed that allow ingrowth by fungal hyphae but not roots. Unfortunately, though, it is difficult to prevent ON from diffusing to or from the core. Hobbie & Hobbie (2006) recently used natural abundance of ¹⁵N in plants and fungi to estimate that ectomycorrhizal fungi are responsible for 61-86% of N uptake by plants in arctic tundra. However, these calculations require that all pools of available N in the soil display similar ¹⁵N signatures. This condition may not be met in many ecosystems.

In one of the few studies of microbial uptake of QDs, Kloepfer et al. (2005) observed QD-adenine uptake by wild-type strains of Bacillus subtilis, but not by ade and apt mutants, which had previously displayed reduced ability to acquire adenine. They interpreted their findings as evidence the quantum dot uptake was governed by processing mechanisms for the adenine. In our study, Penicillium solitum, Poa annua, and AMF did not take up QDs that were unbound to a substrate, nor did they take up unbound QDs in solution with a substrate but without binding reagent. In this case, it is possible that the fungi and plants were targeting the glycine, arginine, and chitosan for acquisition, and then incidentally taking up the conjugated QDs in the process.

QDs may be used in conjunction with field imaging techniques. Quantum dots of any color will fluoresce under standard UV light. Therefore, QDs are commonly imaged using epifluorescent and confocal microscopy (Voura et al. 2004, Chen 2006). These methods incorporate continuous viewing coupled with light intensity detectors or software, providing quantitative results comparable to fluorescent spectrophotometers (Yezhelyev et al. 2007). Mycorrhizae and plant roots can be imaged in a similar manner using underground minirhizotron cameras. Minirhizotron cameras are commonly fitted with an UV light source and can take photos of 4 μm resolution (Hendrick and Pregitzer 1996). We did not quantitatively examine QD uptake in this study. However, we did use minirhizotron imaging to confirm ON uptake by fungal hyphae in soil. Moreover, we found that QDs can be used with portable handheld UV lights to observe uptake in grass shoots with the naked eye. This method would allow a researcher to apply QD-labeled nutrients to natural systems and non-destructively determine uptake among plants in real-time.

In applying QDs in ecological studies, we must consider two major concerns: artifacts related to QD size, and potential toxicity of heavy metals. Currently, most commercial polymer QDs are fairly large after conjugation—roughly the size of small proteins. Quantum dot size can influence uptake dynamics of labeled compounds. Kloepfer et al. (2005) found that QDs larger than 5 nm diameter were not taken up by certain bacterial cells. However, the field of QD technology is rapidly advancing, and some commercial companies now offer polymer QDs as small as 2 nm, equivalent to the size of two nucleic acids (NNT, Ontario, Canada). Smaller QDs could be ideal candidates to address ecological questions comparing multiple microorganisms such as bacteria and fungi. Additionally, heavy metal toxicity from QDs has been demonstrated in bacteria and human cell lines (e.g., Kloepfer et al. 2005, Cho et al. 2007). However, little is known about QD effects to the environment. Most commercial QDs contain cores of cadmium, a known carcinogen. Although, cadmium cores are protected in zinc sulfide and polymer coatings, strict safety precautions must be considered for QD use in environmental studies. As a possible alternative, iron- and silica-based QDs are currently in development (Zheng et al. 2005, Buehler et al. 2006, Lee et al. 2007). Since they contain no cadmium, toxicity should be less of a concern. These versions of QDs may be particularly useful for ecological applications.

Our study contributes to the growing body of literature demonstrating that AMF may have a more significant effect on ON dynamics than previously believed (Näsholm et al. 1998, Hawkins et al. 2000, Hodge et al. 2001, Rains and Bledsoe 2007). The QD technique provides a simple, inexpensive, and non-destructive measure of mycorrhizal fungi and root uptake in natural systems. By using this technique to trace the uptake of ON molecules, researchers could improve our knowledge of nutrient acquisition by organisms in general, and the role of AMF in plant ON uptake in particular.

### A Nanotechnological Technique to Improve Effectiveness of Active Ingredients Against Agricultural Pests

Applicants have developed the use of nanoparticles to enhance uptake of active ingredients into agricultural pests. Relevant nanoparticles include quantum dots (QDs), iron nanoparticles, magnetic or paramagnetic nanoparticles, ceramic nanoparticles, nanocrystalline silicon, colloidal gold, carbon nanotubes, silver nanoparticles, silicon nanoparticles, and dendrites. Active ingredients include, but are not limited to, fungicides, herbicides, insecticides, and pesticides. Pests include, but are not limited to fungi, rusts, water molds, bacteria, weeds, insects, or nematodes. We have designed nanoparticles that facilitate uptake by the organism, penetration into fungal and plant cells, and unpacking of the active ingredient. In addition, we have developed nanoparticles that direct active ingredients to particular sites within the fungus or plant, and that deter uptake by cells of non-target organisms. In doing so, we have increased efficiency of active ingredients 10-fold.

Nano-Packaging of Fungicides.

Quantum dots and other nanoparticles can be conjugated with active ingredients. In addition, QDs can be bound to various targeting probes that will induce uptake by the fungal pathogen of interest, inhibit uptake by other organisms, or direct the QD to the site of activity of the fungicide (e.g., cytoplasm or vacuole). In this way, QDs act as a “nano-package” of fungicides and target probes that can be fine-tuned to deliver the fungicide precisely where needed.

Quantum Dots Track Chemicals Through Fungi and Plants.

As described above with respect to FIGS. 1A through 4G, fungi and plants actively take up and translocate QD-conjugated amino acids from soil and media. For example, QDs are readily taken up by fungi when the QDs are conjugated with amino acids like glycine and arginine. Moreover, QDs are directed to different locations within the fungus depending on the type of amino acid to which they are bound. Specifically, glycine-bound QDs are translocated to vacuoles within fungal cells (FIG. 2c), whereas arginine-bound QDs remain in the cytoplasm (FIG. 2d). In other words, these amino acids are serving as targeting probes. In addition, QDs conjugated with the amino sugar chitosan were taken up reasonably well by Penicillium fungi (FIG. 2e), but less readily by fungi of the Glomeromycota arbuscular mycorrhizal fungi), which form beneficial relationships with plants. In this way, QDs are targeting particular species of fungi. The published paper is included here as an appendix.

Quantification of Quantum Dot Uptake Under Natural Conditions.

Applicants have completed a field-based study in a boreal forest in Alaska to confirm that fungi and plants will absorb from the soil QDs associated with glycine and chitosan. Furthermore, we established that fungi and plants perform this activity as a means of acquiring nutrients from the soil. Glycine and chitosan contain the nutrient nitrogen, which is required by all living organisms for growth and maintenance. Thus, the present invention takes advantage of this activity by using targeting probes that contain nitrogen to induce uptake by organisms. In addition, as described in a subsequent example further below in this patent application, Applicants have developed a method to quantify the number of quantum dot particles within samples. This measurement will facilitate the development and assessment of the efficacy of specific quantum dot applications in accordance with the present invention.

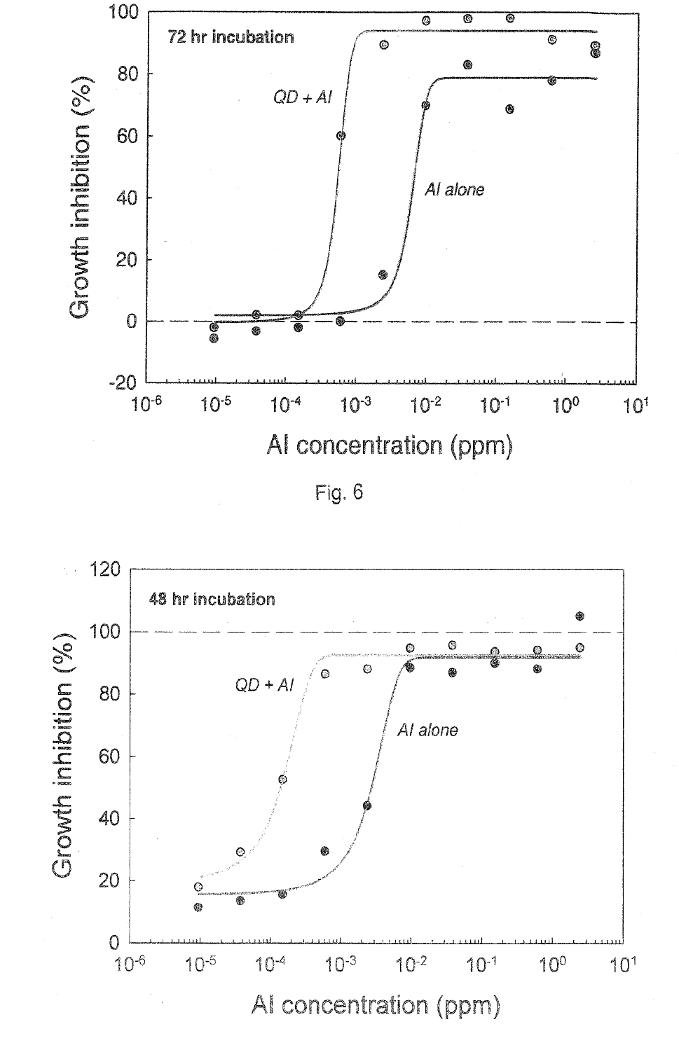

FIGS. 5A through 5B show raster-scanned false color images of QD-chitosan within intracellular arbuscular mycorrhizal (AM) hyphae and a plant root. FIG. 5A is a confocal scan of an Alaskan root colonized with AM hyphae. FIG. 5B is an image with enhanced fluorescent intensities. FIG. 5C is a raster scan (33.8×zoom) of QD-chitosan (616-624 nm emission) within intracellular AM hyphae, which shows an average of 6,241 QD particles per μm³ AM tissue were quantified across the sample image.

Improvement in Effectiveness of Fungicides Via Conjugation with Quantum Dots and Targeting Probes.

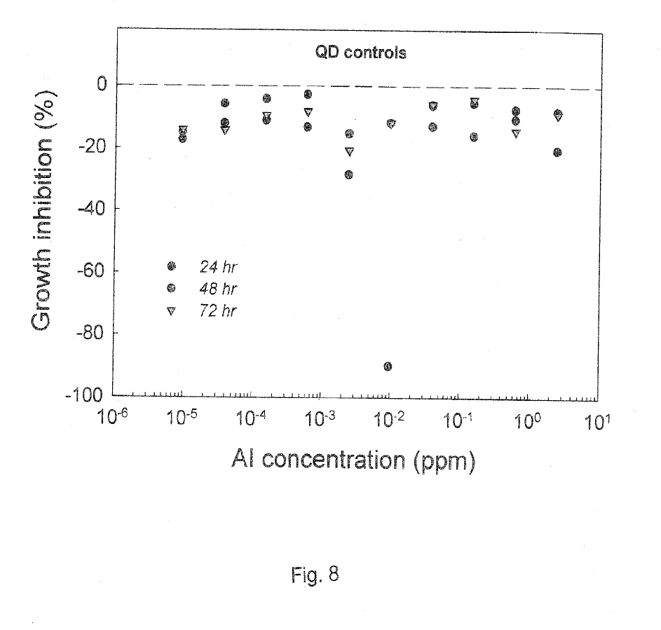

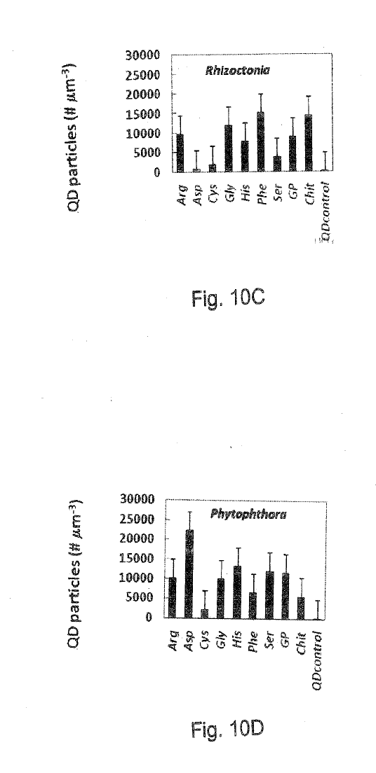

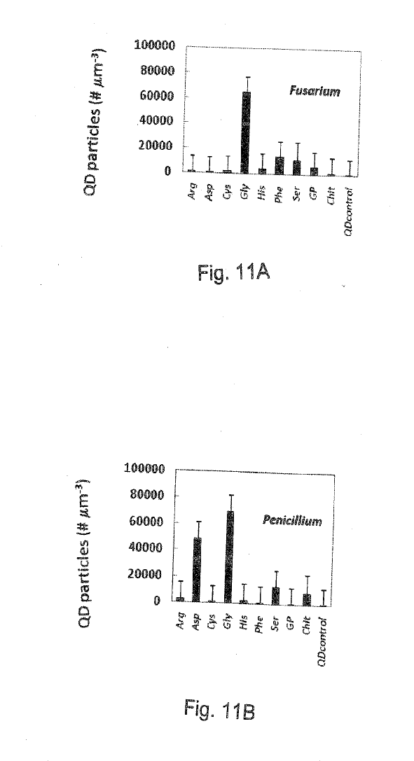

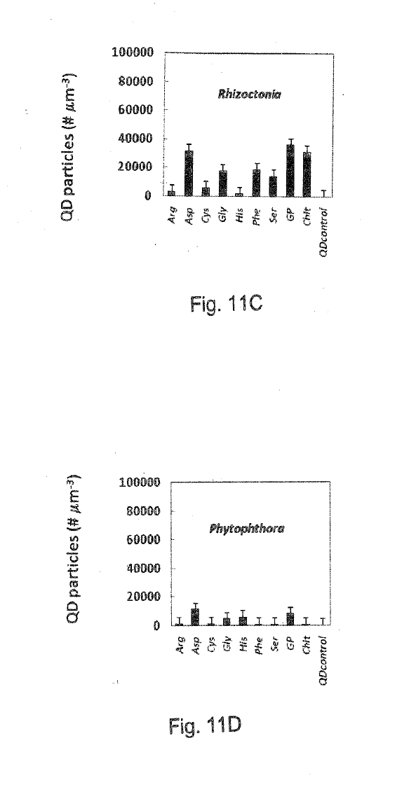

Applicants' QD-bound fungicides are 10 times more effective than unbound fungicides in in vitro assays of growth/germination inhibition, for a broad selection of fungal taxa and two fungicides (Azoxystrobin and Difenoconozole) (FIGS. 9 & 10). Moreover, QDs themselves do not appear to be toxic to fungi, as QDs conjugated to glycine but not fungicides do not inhibit growth (FIGS. 6A through 6F). Applicants have used confocal microscopy to confirm that QD-bound fungicides are taken up by these fungi, as shown in FIGS. 9A through 9C. Also, Applicants have quantified variations in the effectiveness of several targeting probes in directing QDs to vacuoles versus cytoplasm in fungal pathogens, as shown graphically in FIGS. 10A-10D and 11A through 11D. Also, Applicants have established the following protocol for conjugation that provides consistent in vitro results:

Protocol For Conjugation Of Qds With Glycine And Active Ingredients

Vive Nano carboxyl QDs (530 nm emission, 3 nm diameter) are mixed
with PAH and excess EDC (@ 2:1 PAH to QD) in borate buffer.
After 2 hours glycine and excess EDC (@ 33:1 glycine to QD) are added
and the solution is mixed for 2 hours.
Solution is washed with borate buffer in an ultra centrifugal filter.
Solution is diluted with borate buffer (20% DMSO, 5% pluronic).
Final ratios were 4:1 (active ingredient to QD).

### Quantum Dots Highlight Uptake of Organic Nitrogen by Mycorrhizal Fungi Under Field Conditions

Applicants have developed a quantitative nanotechnological technique to directly assess that arbuscular mycorrhizal and ectomycorrhizal fungi can take up labile and recalcitrant organic nitrogen in a natural ecosystem, and that nitrogen availability may regulate this process.

Organic nitrogen (ON) breakdown in soil has recently received recognition as a potential rate-limiting step in nitrogen cycling. The contribution of mycorrhizal fungi (root symbionts) to plant acquisition of ON is one possible mechanism underlying this paradigm. However, mycorrhizal fungi uptake of ON in natural systems has traditionally been difficult to test. We developed a novel quantitative nanotechnological technique to determine in situ that ON uptake can occur much more broadly among mycorrhizal taxa than has previously been assumed. Specifically, arbuscular mycorrhizal (AM) as well as ectomycorrhizal (ECM) fungi acquired recalcitrant ON. Moreover, N enrichment of soil reduced plot-scale uptake. Since most plants host AM or ECM fungi, mycorrhizal use of ON could widely influence plant productivity, especially where N availability is relatively low.

Recent decades have seen a shift in the dominant paradigm describing key controls over N cycling within ecosystems. Classically, mineralization of organic N to ammonium-N or nitrate-N by microbes was thought to determine levels of N available for plant uptake, and, ultimately, net primary productivity. However, findings that plants can acquire soil ON have contributed to the development of a new paradigm in which the depolymerization of relatively large ON compounds in the soil by extracellular enzymes is the major rate-limiting step in the N cycle. An assumption of the new paradigm is that plants can effectively access the smaller ON compounds released from depolymerization, even though physiological attributes of decomposer microbes suggest that they should out-compete plants for these products. Mycorrhizal fungi, which transfer nutrients to their host plants in exchange for photosynthate-C, might improve plants' ability to compete for ON. Nevertheless, the degree to which mycorrhizal fungi acquire ON in natural systems remains a matter of debate, and has traditionally been challenging to determine under field conditions. Applicants used raster image correlation spectroscopy (RICS) and quantum dots (QDs) to address the extent to which major groups of mycorrhizal fungi access labile and recalcitrant soil ON under high and low N conditions in situ. We found that AM fungi (Glomeromycota) could readily access recalcitrant as well as labile ON, even though this phylum has traditionally been thought to take up labile (if any) ON. Since AM fungi are associated with almost 75% of plant species worldwide, this process may influence ON cycling in many terrestrial ecosystems. Ectomycorrhizal (ECM) fungi (Ascomycota and Basidiomycota) also acquired labile and recalcitrant ON, which was consistent with previous characterizations of this group. These data document recalcitrant ON uptake by AM fungi in situ and the successful application of QDs as a quantitative assessment of biogeochemical processes.

Mycorrhizal fungi potentially contribute N to plants from both recalcitrant (i.e., relatively large and complex) and labile (i.e., relatively small and simple) ON sources. Some mycorrhizal fungi can directly take up labile ON forms such as amino acids and small peptides via permeases. Larger, more recalcitrant molecules must be metabolized by extracellular enzymes within the soil solution prior to mycorrhizal uptake. Ectomycorrhizal fungi can release a broad array of extracellular enzymes, and they have been shown to access ON in boreal soils. In contrast, AM fungi have largely been thought to specialize primarily on inorganic nutrients. It has been reported that AM host plants can take up amino acids from boreal forest soil. However, their isotopic labeling technique could not determine whether AM fungi contributed to the uptake.

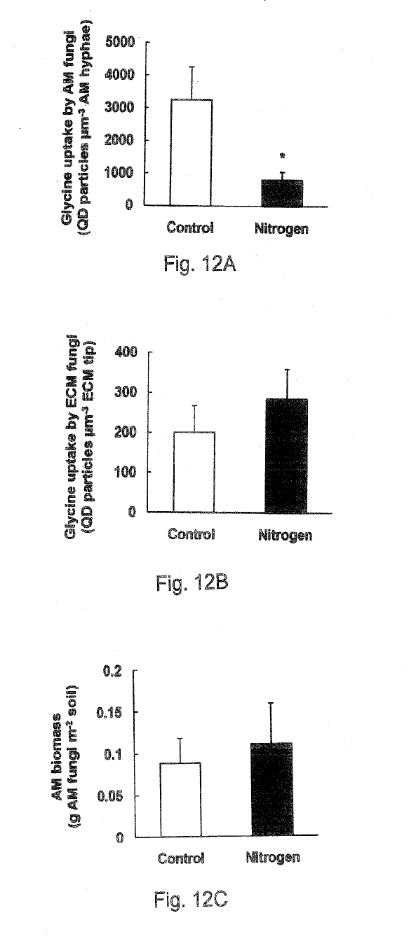

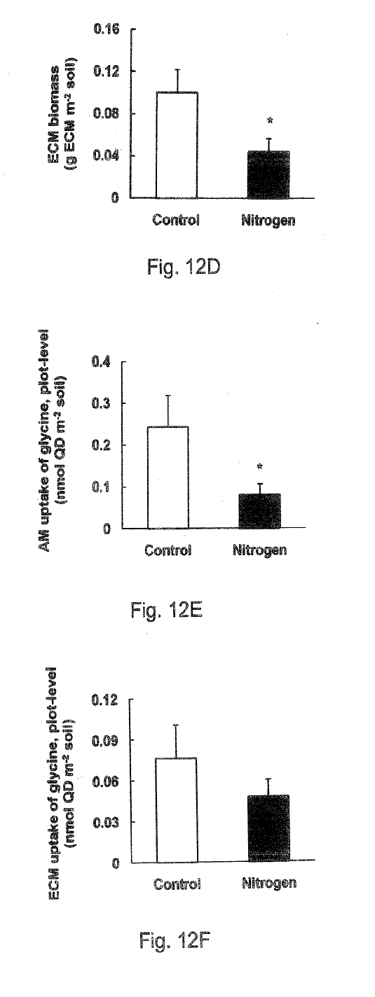

In the present invention, Applicants have applied QDs in conjunction with RICS in a boreal forest with low baseline N availability to examine ON acquisition by mycorrhizal fungi in response to N addition. Glycine (the smallest of the amino acids) was used for labile ON and chitosan (a large, complex, N-containing polysaccharide) for recalcitrant ON. It was determined that AM and ECM fungi each took up labile and recalcitrant ON (FIGS. 12A-12D and 13A-13D). In addition, decreases in plot-level uptake of recalcitrant ON by ECM fungi were observed (FIG. 13D), and of labile ON by AM fungi (FIG. 12A). In each case, N addition could reduce plant acquisition of ON that would otherwise occur via uptake by mycorrhizal fungi.

It has traditionally been assumed that AM fungi do not exploit ON—especially recalcitrant ON—in soils. Applicants' findings of QD-chitosan and QD-glycine uptake by AM fungi in the field challenge this pre-existing view and suggest that AM fungi may play a greater role in ON use than previously thought. Nonetheless, Applicants' results are consistent with others who have recently observed ON uptake by AM fungi in laboratory settings. In addition, others have reported gene expression of amino acid permeases in AM fungi. Furthermore, some AM species can produce chitinases, which are commonly used to defend against root pathogens. Aseptic cultures of AM fungi are reportedly able to access QD-chitosan.

Arbuscular mycorrhizal fungi displayed significantly lower specific uptake rates (i.e., per unit biovolume) of labile ON under N fertilization (FIG. 12C). This result suggests that AM fungi adjust uptake rates of these substrates potentially to curtail costs of N acquisition. Since permeases and extracellular enzymes can require up to 17% of C and 6% of N resources for mycorrhizal fungi and constitute as much as 50% of cell membrane mass, then mycorrhizal fungi should prefer inorganic forms when they are available. This process could be regulated genetically. It has recently been reported that AM fungi expressed fewer amino-acid transporters when inorganic N sources were abundant. The decrease in specific uptake rates of labile ON by AM fungi led to a decline in plot-scale uptake rates, even though AM abundance did not change significantly (FIGS. 12A and 12C).

In contrast, ECM biomass declined under N fertilization (FIG. 12D), which is consistent with previous studies in N fertilized boreal forests and elsewhere. These results coincide with plant allocation theory, which predicts that plants should invest less C in their mycorrhizal symbionts when nutrients are abundant. The reduction in plot-level uptake of recalcitrant ON by ECM fungi under N fertilization was driven primarily by the decrease in ECM biomass (FIGS. 12D, 13D), as there were no significant changes in specific uptake rates for this group (FIGS. 12B, 13B). Some ECM prefer ON to inorganic N in laboratory cultures. Our results suggest that the ECM fungi in this boreal forest may have similar preferences, as there were no significant changes in specific uptake rates of ON under inorganic N addition (FIG. 12B, 13B).

These data confirm AM and ECM uptake of labile and recalcitrant ON in situ. These results support the mycorrhizal component of the new paradigm and provide N conditions under which the paradigm may operate. For instance, mycorrhizal uptake of ON is apparent but less prevalent under N enrichment compared to baseline conditions. Our results suggest that shifts in mycorrhizal abundance (as seen with ECM fungi) and specific uptake of ON (as seen with AM fungi) may be two mechanisms that drive this pattern. Thus, if mycorrhizal fungi process significant amounts of ON in ecosystems—as has been suggested—this pathway could be a crucial step in plant N acquisition and ultimately productivity; increasingly so in areas that contain lower levels of available N.

Applicants used RICS as a novel approach for QD quantification. Since autofluorescence does not correlate with RICS, we observed a consistently high signal-to-noise ratio in our samples. Moreover, we were able to detect as few as four QDs per biovolume sample. Thus, QDs would be especially useful in microscale studies where sample sizes or fluxes are too small for isotopic analysis. For instance, it is often difficult to isolate enough microbial (including AM) tissue from the soil to perform isotopic measurements. In contrast, QDs can provide quantitative, real-time, spatially-explicit information within microscopic structures. In addition, these techniques do not require any costly or major apparati, and can be performed on most standard confocal microscopes. Indeed, the applicability of QDs to ecosystems research may extend beyond the specific, non-limiting examples described in this patent application. For example, QDs and methods of the present invention may be employed to examine a variety of ecological processes, including competition between bacteria and fungi for organic substrates, nutrient allocation and transport within plants, or herbivory or predation rates. In addition, they could be conjugated with a broad array of organic compounds to measure total contributions of mycorrhizal fungi to C and N cycling within ecosystems.

Quantum dots of the present invention can be conjugated with almost any chemical compound with a known structure. Melting points, solubility, and volatility would not restrict the use of quantum dots. They should also be compatible with agricultural carriers, as quantum dots are currently used in solutions containing nutrients typical of common fertilizers.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

1. A system comprising a quantum dot or nanoparticle in combination with at least one microbicidal substance, wherein the quantum dot is operative to deliver the microbial substance to a target cell or organism within a plant or animal subject.

2. A system according to claim 1 wherein the quantum dot or nanoparticle comprises a quantum dot having a core and a shell.

3. A system according to claim 2 wherein the core comprises cadmium selenide.

4. A system according to claim 2 wherein the shell comprises a zinc sulfide.

5. A system according to claim 1 wherein the quantum dot or nanoparticle is substantially free of cadmium.

6. A system according to claim 5 wherein the quantum dot or nanoparticle is iron-based.

7. A system according to claim 6 wherein the quantum dot or nanoparticle is silica-based.

8. A system according to claim 1 wherein the quantum dot or nanoparticle further comprises a polymer coating.

9. A system according to claim 1 wherein the microbicidal substance comprises a fungicide.

10. A system according to claim 9 wherein the fungicide is selected from the group consisting of: dimethomorph; dimethomorph+mancozeb; fosetyl-aluminum; metalaxl; azoxystrobin; mefenoaxm; potassium bicamonate; copper sulfate; PCMB; DCNA; chlorothalonil; pyraclostrobin; copper oxychloride sulfate; captan; copper hydroxide; copper ammonium carbonate; copper sulfate; mancozeb+copper sulfate; copper sulfate; cymoxanil; mancozeb; chlorothalonil; boscalid; trifloxystrobin; tebuconazole; dimethomorph; mono-, dibasic sodium salts of phosphorus acid; zoxamide/mancozeb; pyraclostrobin; iprodione; copper hydroxide; bacillus subtilis; sulfur; maneb; copper hydroxide/mancozeb; mancozeb; fludioxonil; thiabendazole; sulfur; myclobutanil; copper hydroxide; mancozeb; mono-, dibasic sodium, potassium, and ammonium phosphites; fluopicolide; propamocarb; boscalid+pyraclostrobin; triflumizole; mono-, dibasic sodim, potassium and ammonium phosphites; propiconazole; azoxystrobin; azoxystrobin/chlorothalonil; quinoxyfen; azoxystrobin/propiconzanole; fenamidone; mandipropamid; mandipropamid/difenoconazole; mefenoxam/macozeb; mefenoxam/copper hydroxide; mefenoxam/PCNB; mefenoxam; mefenoxam; vinclozolin; iprodine; pyrimethanil; Bacillus subtillis; Gliocladium virens; sulfur; cypodinil/fludioxonil; metalaxyl+PCNB+Bacillus subtillis; famoxadone+cymoxanil; famoxadone+cymoxanil; dichloropropene/chloropicrin; copper salts of fatty & rosin acid; PCNB; Sulfur; Thiram; Propiconazole; thiophanate-methyl; nemm oil; Mefenoxam; metam-sodium and ziram.

11. A system according to claim 1 further comprising a targeting substance, compound, moiety or group that facilitates uptake of the microbicidal substance by the target cell or organism or directs the quantum dot to an intended site of fungicide activity.

12. A system according to claim 11 wherein the targeting substance, compound, moiety or group comprises an amino acid.

13. A system according to claim 1 wherein the target cell or organism comprises a fungus.

14. A method for treating or deterring microbial infestation or growth in a plant or animal subject, said method comprising the step of administering to the subject a quantum dot or nanoparticle combined with at least one microbicidal substance, wherein the quantum dot or nanoparticle is operative to deliver the microbial substance to a target cell or organism within the plant or animal subject.

15. A method according to claim 14 wherein the subject is an agricultural plant.

16. A method according to claim 15 wherein the plant is infested with a fungal organism and wherein the microbicidal substance comprises a fungicide.

17. A method according to claim 16 wherein the target cell or organism is the fungal organism.

18. A method according to claim 16 wherein the fungicide is selected from the group consisting of: dimethomorph; dimethomorph+mancozeb; fosetyl-aluminum; metalaxl; azoxystrobin; mefenoaxm; potassium bicamonate; copper sulfate; PCMB; DCNA; chlorothalonil; pyraclostrobin; copper oxychloride sulfate; captan; copper hydroxide; copper ammonium carbonate; copper sulfate; mancozeb+copper sulfate; copper sulfate; cymoxanil; mancozeb; chlorothalonil; boscalid; trifloxystrobin; tebuconazole; dimethomorph; mono-, dibasic sodium salts of phosphorus acid; zoxamide/mancozeb; pyraclostrobin; iprodione; copper hydroxide; bacillus subtilis; sulfur; maneb; copper hydroxide/mancozeb; mancozeb; fludioxonil; thiabendazole; sulfur; myclobutanil; copper hydroxide; mancozeb; mono-, dibasic sodium, potassium, and ammonium phosphites; fluopicolide; propamocarb; boscalid+pyraclostrobin; triflumizole; mono-, dibasic sodim, potassium and ammonium phosphites; propiconazole; azoxystrobin; azoxystrobin/chlorothalonil; quinoxyfen; azoxystrobin/propiconzanole; fenamidone; mandipropamid; mandipropamid/difenoconazole; mefenoxam/macozeb; mefenoxam/copper hydroxide; mefenoxam/PCNB; mefenoxam; mefenoxam; vinclozolin; iprodine; pyrimethanil; Bacillus subtillis; Gliocladium virens; sulfur; cypodinil/fludioxonil; metalaxyl+PCNB+Bacillus subtillis; famoxadone+cymoxanil; famoxadone+cymoxanil; dichloropropene/chloropicrin; copper salts of fatty & rosin acid; PCNB; Sulfur; Thiram; Propiconazole; thiophanate-methyl; nemm oil; Mefenoxam; metam-sodium and ziram.

19. A method according to claim 16 wherein the fungal organism comprises an organism selected from the group consisting of: organisms of class Ascomycetes; organisms of class Basidiomycetes; Fusarium; Thielaviopsis; Penicillium; Verticillium; Magnaport; Rhizoctonia; Phakospora pachyrhizi; Puccinia; oomycetes; organisms of the genus Phytophthora; Pythium and Phytophthora.

20. A method according to claim 14 wherein the quantum dot or nanoparticle comprises a quantum dot having a core and a shell

21-27. (canceled)
